(19) United States
(12) Patent Application Publication
(10) Pub. No.: US 2017/0086779 A1
(43) Pub. Date: Mar. 30, 2017

(54) EATING AND DRINKING ACTION DETECTION APPARATUS AND EATING AND DRINKING ACTION DETECTION METHOD

(71) Applicant: FUJITSU LIMITED, Kawasaki-shi (JP)

(72) Inventors: Akira Kamano, Kawasaki (JP); Takeshi OTANI, Kawasaki (JP); Yohei KISHI, Kawasaki (JP)

(73) Assignee: FUJITSU LIMITED, Kawasaki-shi (JP)

(21) Appl. No.: 15/259,149

(22) Filed: Sep. 8, 2016

(30) Foreign Application Priority Data
Sep. 24, 2015 (JP) 2015-186638

(51) Int. Cl. A61B 7/04; A61B 5/00; A61B 5/11

(52) U.S. Cl. CPC A61B 7/04; A61B 5/11; A61B 5/4205; A61B 5/7275

(57) ABSTRACT

The eating and drinking action detection apparatus: acquires vibration produced from inside of a body of a subject and generates a vibration signal corresponding to the vibration; divides the vibration signal into each frame to calculate power of the vibration signal for each frame; determines, for each frame, whether the frame is a stationary signal having a periodicity or a non-stationary signal having no periodicity; detects, based on the power of each frame and a determination result for each frame whether the frame is the stationary signal or the non-stationary signal, a period of the non-stationary signal being continued while the power of the vibration signal is equal to or larger than a power threshold, acquires a continuation time of the period; and determines, based on the continuation time, whether the subject performed swallowing or mastication in the period of the non-stationary signal being continued.

### CROSS-REFERENCE TO RELATED APPLICATION

This application is based upon and claims the benefit of priority of the prior Japanese Patent Application No. 2015-186638, filed on Sep. 24, 2015, and the entire contents of which are incorporated herein by reference.

### FIELD

The embodiments discussed herein are related to an eating and drinking action detection apparatus and an eating and drinking action detection method for detecting an action of eating and drinking of a subject.

### BACKGROUND

Research has been conducted on analyzing an action or state of a person on the basis of a signal such as sound or the like uttered by the person. As an example of the research, techniques for detecting an action of eating and drinking, e.g., swallowing or mastication, of a subject on the basis of vibration produced from the inside of the body of the subject have been proposed (for example, see Japanese Laid-open Patent Publication No. 2008-61790, Japanese Laid-open Patent Publication No. 2009-279122, and Japanese Laid-open Patent Publication No. 2012-196284).

For example, Japanese Laid-open Patent Publication No. 2008-61790 discloses an utterance/eating-and-drinking state detection system that determines whether a state is an utterance state or an eating and drinking state, on the basis of output data acquired by arithmetic processing of an output signal from an internal-body sound microphone that detects internal-body sound. The utterance/eating-and-drinking state detection system determines that a state is an eating and drinking state when the power of a fundamental frequency component of output data during a predetermined period is smaller than a first threshold and the power of the output data during the predetermined period is equal to or larger than a second threshold. Further, the utterance/eating-and-drinking state detection system calculates an occurrence frequency of the eating and drinking state in a certain period, and determines whether a state is a mastication state or a swallowing state. In this determination, the utterance/eating-and-drinking state detection system determines that the state is a mastication state, when the occurrence frequency is high, while determining that the state is a swallowing state, when the occurrence frequency is low.

The state detection apparatus disclosed in Japanese Laid-open Patent Publication No. 2009-279122 separates a vibration signal of a living body into a body movement signal and an audio signal and detects swallowing of the subject on the basis of a swallowing sound detected from the audio signal and swallowing movement of the throat detected from the body movement signal.

Further, the mastication detection apparatus disclosed in Japanese Laid-open Patent Publication No. 2012-196284 measures a mastication sound, obtains an outline of a power transition of the measured mastication sound in the time direction, and mastication is determined on the basis of the outline.

### SUMMARY

When eating and drinking, a person may masticate and swallow. In such a case, the technique disclosed in Japanese Laid-open Patent Publication No. 2008-61790 may fail to identify swallowing with mastication, since a mastication state and a swallowing state are included in a certain period and hence swallowing is erroneously identified as mastication. It is not a target of the technique disclosed in Japanese Laid-open Patent Publication No. 2009-279122 and the technique disclosed in Japanese Laid-open Patent Publication No. 2012-196284 to identify mastication with swallowing.

According to one embodiment, an eating and drinking action detection apparatus is provided. The eating and drinking action detection apparatus includes: a vibration sensor configured to acquire vibration produced from inside of a body of a subject and generate a vibration signal corresponding to the vibration; and a processor. The processor is configured to: divide the vibration signal into each frame with a predetermined time length to calculate power of the vibration signal for each frame; determine, for each frame, whether the frame is a stationary signal having a periodicity or a non-stationary signal having no periodicity; detect, based on the power of each frame and a determination result for each frame whether the frame is the stationary signal or the non-stationary signal, a period of the non-stationary signal being continued while the power of the vibration signal is equal to or larger than a power threshold; acquire a continuation time of the period; and determine, based on the continuation time, whether the subject performed swallowing or mastication in the period of the non-stationary signal being continued.

The object and advantages of the invention will be realized and attained by means of the elements and combinations particularly pointed out in the claims.
It is to be understood that both the foregoing general description and the following detailed description are exemplary and explanatory and are not restrictive of the invention, as claimed.

### BRIEF DESCRIPTION OF DRAWINGS

FIG. 1 is a drawing illustrating an example of an audio signal including a mastication sound and a swallowing sound.

FIG. 2 is a schematic block diagram of an eating and drinking action detection apparatus according to a first embodiment.

FIG. 3 is a functional block diagram of a processing unit according to the first embodiment.

FIG. 4 is an operational flowchart of stationarity determination processing on the basis of an autocorrelation sequence.

FIG. 5 is an operational flowchart of continuation time measurement processing of a non-stationary signal.

FIG. 6 is an operational flowchart of eating and drinking action detection processing.

FIG. 7 is a drawing illustrating an example of a detection result when the eating and drinking action detection processing according to the present embodiment is applied to an audio signal acquired as a vibration signal.

FIG. 8 is a functional block diagram of a processing unit according to a second embodiment.

FIG. 9 is an operational flowchart of threshold control processing.

FIG. 10 is a functional block diagram of a processing unit of an eating and drinking action detection apparatus according to a third embodiment.

FIG. 11 is a functional block diagram of a processing unit of an eating and drinking action detection apparatus according to a fourth embodiment.

### DESCRIPTION OF EMBODIMENTS

An eating and drinking action detection apparatus is described below with reference to the drawings. The eating and drinking action detection apparatus detects an action of eating and drinking of a subject, in particular mastication and swallowing by analyzing a vibration signal corresponding to vibration produced by the subject.

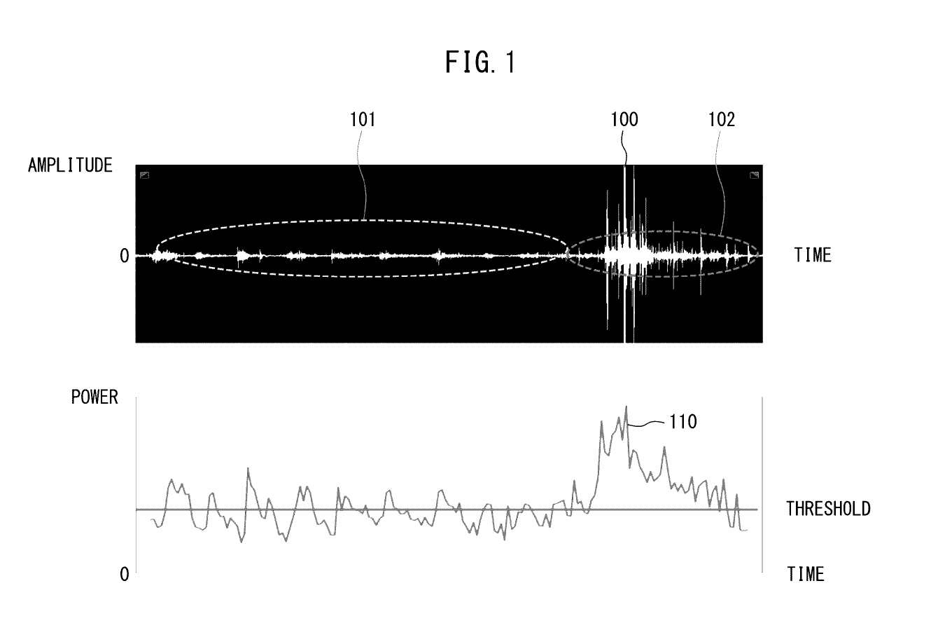

FIG. 1 is a drawing illustrating an example of an audio signal including a mastication sound and a swallowing sound. In FIG. 1, a horizontal axis represents time and a vertical axis represents amplitude of an audio signal. A signal 100 represents the audio signal. In FIG. 1, a section 101 corresponds to a masticating section, and, on the other hand, a section 102 corresponds to a swallowing section. A graph 110 depicted under the audio signal 100 represents a temporal change of power of the audio signal 100.

A swallowing sound is sound produced by an impact due to a motion of muscles in the pharynx, and by a fricative when food passes through a throat, or by alternation of air and food. The swallowing sound continues for 500 milliseconds to 800 milliseconds. On the other hand, a mastication sound is sound produced when food is crushed, and the continuation time of the mastication sound is shorter than the swallowing sound. For example, the mastication sound continues for 100 milliseconds to 300 milliseconds. As depicted in the audio signal 100 and the graph 110, neither the mastication sound nor the swallowing sound has periodicity, i.e., a pitch, and either sound is a non-stationary sound which has a narrow autocorrelation range in comparison with an utterance sound. When comparing the mastication sound with the swallowing sound, a continuation period of a power above a certain level in the mastication sound is relatively shorter than a continuation period of a power above the certain level in the swallowing sound.

Therefore, the eating and drinking action detection apparatus detects a period during which the vibration signal corresponding to vibration produced by the subject has a power above a certain level and a non-stationary signal continues, as a swallowing or masticating period. The eating and drinking action detection apparatus identifies whether the period corresponds to mastication or swallowing in response to the length of the period.

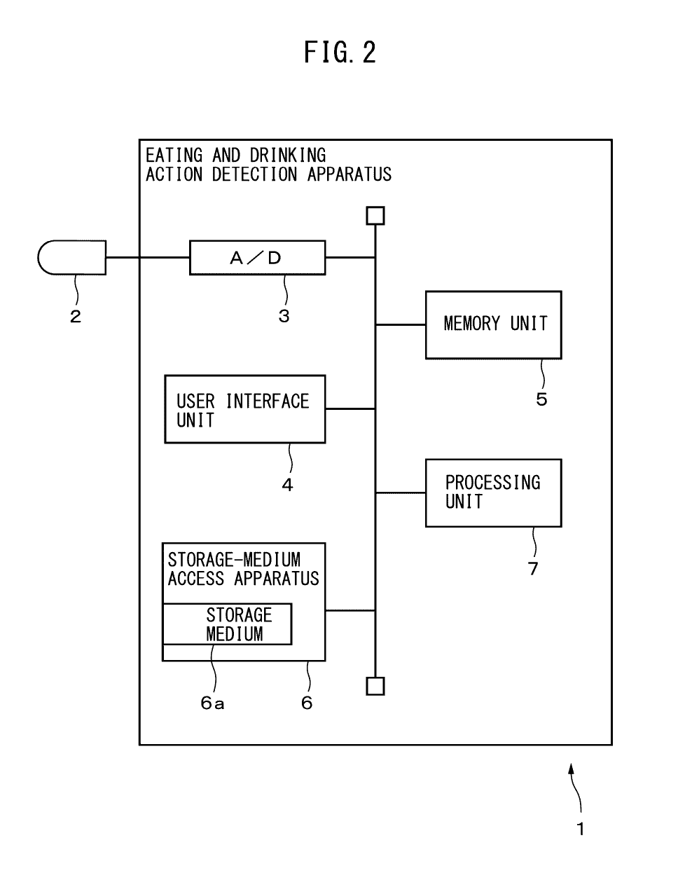

FIG. 2 is a schematic block diagram of an eating and drinking action detection apparatus according to a first embodiment. The eating and drinking action detection apparatus 1 is implemented, for example, as a mobile phone, a smartphone, a tablet, or a computer. The eating and drinking action detection apparatus 1 includes a human body vibration acquisition unit 2, an analog-to-digital conversion unit 3, a user interface unit 4, a memory unit 5, a storage-medium access apparatus 6, and a processing unit 7. The eating and drinking action detection apparatus 1 may further include a communication unit (not shown) for communicating with other equipment by wire or by radio.

The human body vibration acquisition unit 2 acquires vibration produced from the inside of the body of the subject, especially vibration of a muscle or a bone in a mouth or around a throat, or sound due to the vibration. The human body vibration acquisition unit 2 generates an electrical signal corresponding to the vibration as the vibration signal. For the purpose, the human body vibration acquisition unit 2 includes, for example, a microphone, a pharynx microphone, a bone conduction microphone, a pickup, an accelerometer, a pressure sensor, a video camera, or a sensor, such as a myoelectric sensor, which can acquire vibration produced from inside the body or the sound due to the vibration. The sensor is embedded in a necklace, an ear hooking, a patch, an earphone, a headphone, or clothing, to be put on, for example, around the neck of the subject, or on the chest, the head or the face. When the sensor is a video camera, the video camera is installed toward the neck or chest of the subject. Then, in an image signal obtained by the video camera, the vibration signal representing the vibration of a muscle or a bone inside the mouth or around the throat is acquired from change of luminance values of pixels or the like in which the neck or chest of the subject is photographed.

The human body vibration acquisition unit 2 outputs the generated vibration signal to the analog-to-digital conversion unit 3.

In a modified example, the human body vibration acquisition unit 2 may be provided separately from a main body of the eating and drinking action detection apparatus, and may transmit an acquired signal to the main body of the eating and drinking action detection apparatus by radio communication. In this case, the human body vibration acquisition unit 2 may be implemented, for example, in a wireless terminal in accordance with a standard corresponding to the Body Area Network, and the wireless terminal may be attached to the subject. The main body of the eating and drinking action detection apparatus may be fixedly installed within a range communicable with the wireless terminal. The wireless terminal may transmit the generated vibration signal to the main body of the eating and drinking action detection apparatus sequentially. Alternatively, the wireless terminal including the human body vibration acquisition unit 2 may store the generated vibration signal for a certain period, and may transmit the stored vibration signal to the main body of the eating and drinking action detection apparatus at the timing when radio communication with the main body of the eating and drinking action detection apparatus is available.

The analog-to-digital conversion unit 3 includes, for example, an amplifier and an analog-to-digital converter. The analog-to-digital conversion unit 3 amplifies by the amplifier the vibration signal received from the human body vibration acquisition unit 2. Then, the analog-to-digital conversion unit 3 samples the amplified vibration signal at a predetermined sampling period by the analog-to-digital converter to generate a digitized vibration signal. Hereinafter, the digitized vibration signal is simply referred to as a vibration signal for convenience. The analog-to-digital conversion unit 3 outputs the vibration signal to the processing unit 7.

The user interface unit 4 includes a touch panel, for example. The user interface unit 4 generates an operation signal corresponding to an operation of a user, such as a signal for instructing a start of a vibration signal analysis or a signal for displaying the result of the vibration signal analysis, and outputs the operation signal to the processing unit 7. The user interface unit 4 displays the result of the vibration signal analysis and the like in accordance with the signal for the display received from the processing unit 7. Note that the user interface unit 4 may include separately a plurality of operation buttons for inputting the operation signal, and a display apparatus such as a liquid crystal display.

The memory unit 5 includes, for example, a readable/writable semiconductor memory and a read-only semiconductor memory. The memory unit 5 stores various computer programs and various kinds of data which are used by the eating and drinking action detection apparatus 1. In particular, the memory unit 5 stores various kinds of information used by eating and drinking action detection processing, and the vibration signal to be processed in the eating and drinking action detection processing.

The storage-medium access apparatus 6 is, for example, an apparatus for accessing a storage medium 6a, such as a semiconductor memory card, a hard disk, or an optical storage medium. The storage-medium access apparatus 6 reads, for example, the computer programs stored in the storage medium 6a to be executed on the processing unit 7, and passes the programs to the processing unit 7.

The processing unit 7 includes one or a plurality of processors, a memory circuit, and a peripheral circuit. The processing unit 7 controls entire eating and drinking action detection apparatus 1.

The processing unit 7 executes the eating and drinking action detection processing on the acquired vibration signal to detect an action of eating and drinking such as mastication and swallowing of the subject.

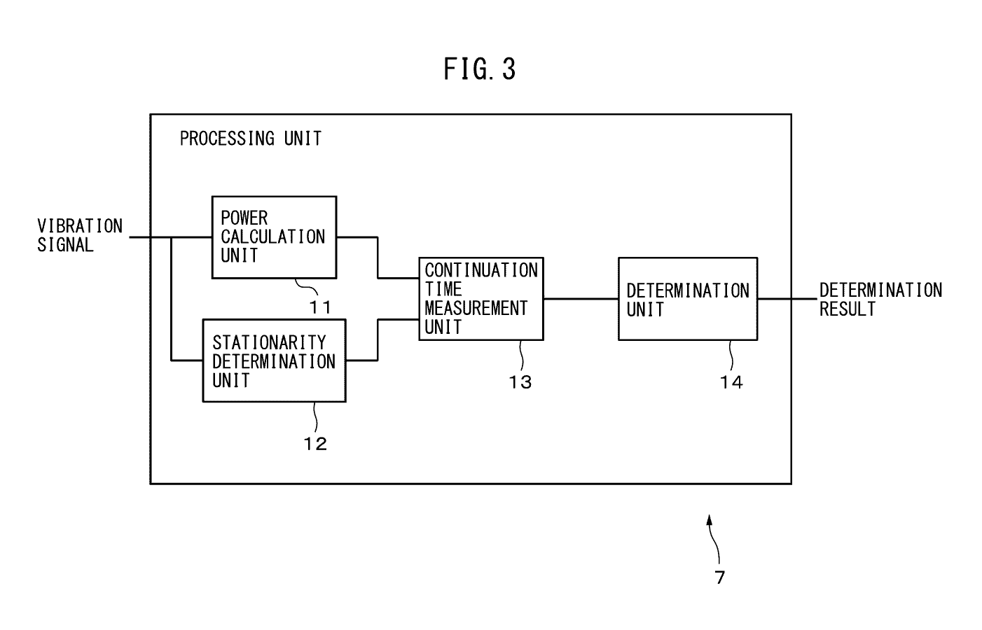

FIG. 3 is a functional block diagram of the processing unit 7 according to the first embodiment. The processing unit 7 includes a power calculation unit 11, a stationarity determination unit 12, a continuation time measurement unit 13, and a determination unit 14. These units included in the processing unit 7 are implemented, for example, as functional modules realized by a computer program executed on a processor or processors. Alternatively, the units included in the processing unit 7 may be implemented in the eating and drinking action detection apparatus 1 as one or a plurality of integrated circuits in which functions of the units are realized separately from the processors included in the processing unit 7.

The power calculation unit 11 divides the vibration signal into each frame with a predetermined length, and calculates the power of a vibration signal for each frame. For example, the power calculation unit 11 calculates power in accordance with a following equation.

$$pow ( t , t + L ) = {\sum }_{i = t}^{t + L} {s ( i )}^{2} \qquad ( 1 )$$

Pow(t, t+L) represents the power of the vibration signal in a frame of length L which starts from time t. In addition, s(i) represents the vibration signal at each sampling point included in the frame. For example, the frame length L is of the order of several milliseconds. and the number of the sampling points included in the frame is of the order of several thousands.

The power calculation unit 11 may perform a temporal frequency transformation such as the Fast Fourier Transform (FFT) on the vibration signal for each frame to calculate a signal in a frequency domain. In this case, the power calculation unit 11 may calculate the power in accordance with a following equation.

$$pow ( t , t + L ) = {\sum }_{f = {f}_{min}}^{{f}_{max}} {S ( f )}^{2} \qquad ( 2 )$$

S (f) is a frequency signal corresponding to the frequency represented by f in the frame which starts from time t. In addition, fmin and fmax represent the lower limit and the upper limit of the frequency utilized for the power calculation, respectively. The lower limit and the upper limit of the frequency may be respectively set to, for example, the values corresponding to the lower limit value and the upper limit value of the frequency component of a swallowing sound or a mastication sound.

The power calculation unit 11 outputs the power of the vibration signal for each frame to the continuation time measurement unit 13. When the stationarity determination unit 12 determines the stationarity of the vibration signal on the basis of the signal in the frequency domain, the power calculation unit 11 may output a signal in the frequency domain for each frame to the stationarity determination unit 12.

The stationarity determination unit 12 determines whether the vibration signal is a stationary signal having a periodicity or a non-stationary signal having no periodicity, for each frame. As described for FIG. 1, the vibration signal due to swallowing and mastication has the lower stationarity than that of an utterance sound, and therefore the stationarity is useful as a feature for identifying the utterance sound with the vibration signal due to swallowing and mastication.

The stationarity determination unit 12 calculates, for example, an autocorrelation sequence of the vibration signal for each frame, and determines whether each frame is the stationary signal or the non-stationary signal on the basis of the autocorrelation sequence.

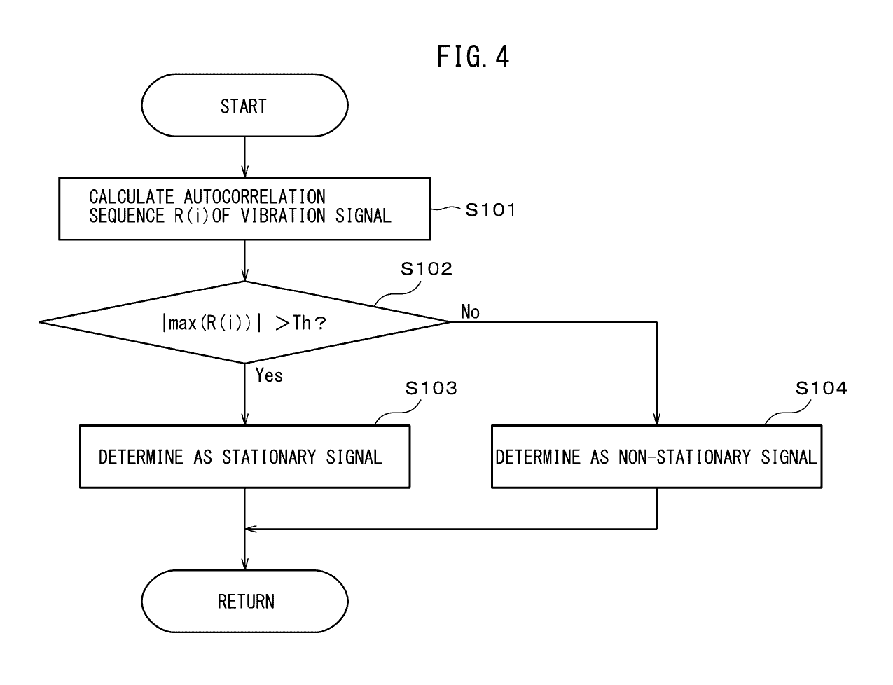

FIG. 4 is an operational flowchart of stationarity determination processing on the basis of an autocorrelation sequence. The stationarity determination unit 12 determines whether the frame is the stationary signal or the non-stationary signal in accordance with the following operational flowchart for each frame.

The stationarity determination unit 12 calculates an autocorrelation sequence of a vibration signal, for example, in accordance with the following equation (step S101).

$$R ( i ) = \frac{1}{L + 1} {\sum }_{j = 0}^{L} s ( t + j ) \cdot s ( t + ( j + i ) \% ( L + 1 ) ) \qquad ( 3 )$$

R(i) (i=1, 2, . . . , L) represents the autocorrelation sequence. Then, the stationarity determination unit 12 determines whether an absolute value |max(R(i))| of the maximum value of autocorrelation values included in the autocorrelation sequence is larger than a threshold Th (step S102). When a target frame of the vibration signal has stationarity, the vibration signal has a certain periodicity, i.e. a pitch, in the frame, which results in a high absolute value of the maximum value of the autocorrelation values. On the other hand, when the target frame of the vibration signal has no stationarity, the vibration signal has no periodicity in the frame, which results in a low absolute value of the maximum value of the autocorrelation values. Therefore, when the absolute value |max(R(i))| of the maximum value of the autocorrelation values is larger than the threshold Th (step S102-Yes), the stationarity determination unit 12 determines that the target frame is the stationary signal (step S103). On the other hand, when the absolute value |max(R(i))| of the maximum value of the autocorrelation values is equal to or smaller than the threshold Th (step S102-No), the stationarity determination unit 12 determines that the target frame is the non-stationary signal (step S104). Note that the threshold Th is set to 0.05, for example. The stationarity determination unit 12 terminates the stationarity determination processing after step S103 or S104.

According to a modified example, the stationarity determination unit 12 may determine whether the target frame of the vibration signal is the stationary signal or the non-stationary signal on the basis of a peak of a spectrum or a cepstrum of the vibration signal, instead of the autocorrelation. For example, the stationarity determination unit 12 squares, for each frequency, the signal in the frequency domain for the target frame of the vibration signal to calculate the spectrum of the vibration signal. Then, the stationarity determination unit 12 detects the peak of the spectrum in the frequencies other than zero. The stationarity determination unit 12 may determine that the target frame is the stationary signal when the peak is higher than a predetermined threshold, and the stationarity determination unit 12 may determine that the target frame is the non-stationary signal when the peak is equal to or lower than the predetermined threshold.

When determining whether the target frame is the stationary signal or the non-stationary signal on the basis of the cepstrum, the stationarity determination unit 12 calculates, for example, an FFT cepstrum, a linear prediction cepstrum, or a Mel frequency cepstrum as the cepstrum. A cepstrum is spectrum envelope information representing the resonance characteristics of the vocal tract of a subject. Therefore, the peak of the cepstrum is relatively high when the target frame includes an utterance sound, whereas the peak of the cepstrum is relatively low when the target frame includes no utterance sound but includes a mastication sound or a swallowing sound. Therefore, the stationarity determination unit 12 may detect the peak of the cepstrum, and determine that the target frame is the stationary signal when the peak is higher than a predetermined threshold. On the other hand, the stationarity determination unit 12 may determine that the target frame is the non-stationary signal when the peak is equal to or lower than the predetermined threshold.

The stationarity determination unit 12 outputs a determination result whether the target frame is the stationary signal or the non-stationary signal for each frame to the continuation time measurement unit 13.

As described above, the mastication sound and the swallowing sound are different in the length of the period as non-stationary signals while respectively holding a power above a certain level. Therefore, the continuation time measurement unit 13 detects the period during which the non-stationary signal having a power above a certain level continues, and measures the continuation time of the period.

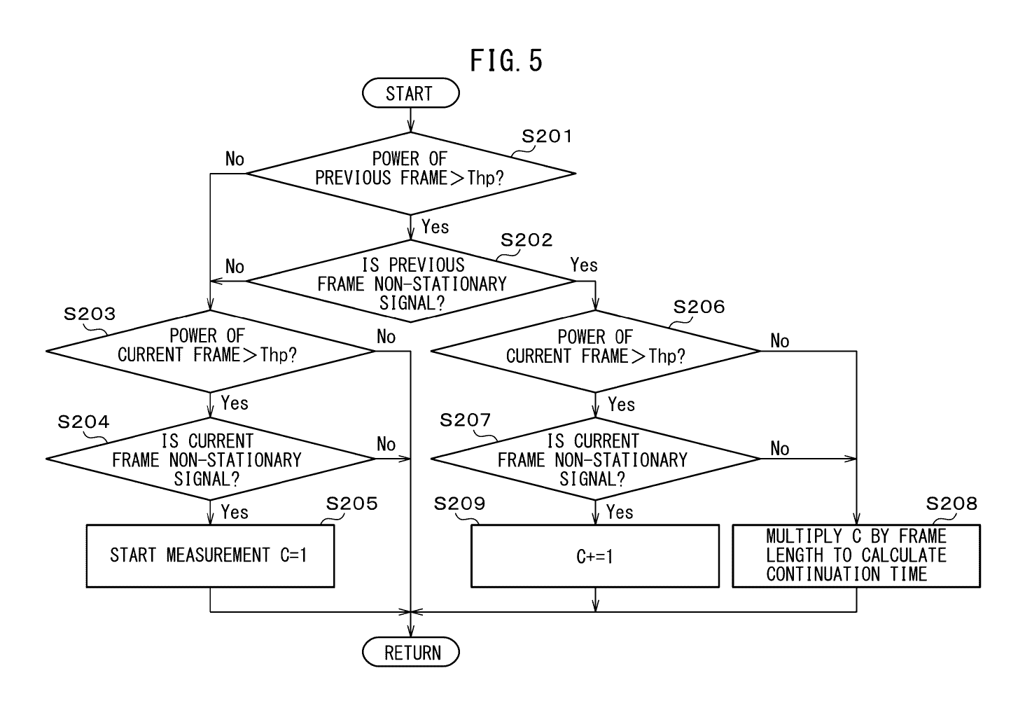

FIG. 5 is an operational flowchart of continuation time measurement processing of a non-stationary signal. The continuation time measurement unit 13 executes continuation time measurement processing of the non-stationary signal for each frame in accordance with the following operational flowchart.

The continuation time measurement unit 13 determines whether power of a previous frame is larger than a predetermined power threshold Thp (step S201). Note that the power threshold Thp is set to, for example, the lower limit value of the power per frame of the vibration signal due to swallowing or mastication. When the power of the previous frame is larger than the power threshold Thp (step S201-Yes), the continuation time measurement unit 13 determines whether the vibration signal of the previous frame is the non-stationary signal (step S202).

When the vibration signal of the previous frame is not the non-stationary signal (step S202-No), or when the power of the previous frame is equal to or less than the power threshold Thp in step S201 (step S201-No), it is expected that the previous frame corresponds to neither mastication nor swallowing. Therefore, in order to determine whether the measurement of the continuation time of the non-stationary signal is started from the current frame, the continuation time measurement unit 13 determines whether the power of the current frame is larger than the power threshold Thp (step S203). When the power of the current frame is equal to or less than power threshold Thp (step S203-No), it is expected that the current frame corresponds to neither mastication nor swallowing. Therefore, the continuation time measurement unit 13 terminates the continuation time measurement processing of the non-stationary signal, without starting the measurement of the continuation time.

On the other hand, when the power of the current frame is larger than the power threshold Thp (step S203-Yes), the continuation time measurement unit 13 determines whether the vibration signal of the current frame is the non-stationary signal (step S204). When the vibration signal of the current frame is not the non-stationary signal (step S204-No), it is expected that the current frame corresponds to neither mastication nor swallowing. Therefore, the continuation time measurement unit 13 terminates the continuation time measurement processing of the non-stationary signal, without starting the measurement of the continuation time. On the other hand, when the vibration signal of the current frame is the non-stationary signal (step S204-Yes), it is assumed that the current frame corresponds to mastication or swallowing. Therefore, the continuation time measurement unit 13 starts the measurement of the continuation time, and sets a count number C of the frame to 1, which indicates that the continuation time of the non-stationary signal is measured (step S205). Then, the continuation time measurement unit 13 terminates the continuation time measurement processing of the non-stationary signal.

When the vibration signal of the previous frame is the non-stationary signal in step S202 (step S202-Yes), the measurement of the continuation time of the non-stationary signal is continued at the time of the previous frame. Therefore, in order to determine whether the measurement of the continuation time is to be terminated at the current frame, the continuation time measurement unit 13 determines whether the power of the current frame is larger than the power threshold Thp (step S206). When the power of the current frame is larger than the power threshold Thp (step S206-Yes), the continuation time measurement unit 13 determines whether the vibration signal of the current frame is the non-stationary signal (step S207).

When the vibration signal of the current frame is not the non-stationary signal (step S207-No), or when the power of the current frame is equal to or less than the power threshold Thp in step S206 (step S206-No), it is assumed that the current frame corresponds to neither mastication nor swallowing. Therefore, the continuation time measurement unit 13 multiplies the count number C at the time of the current frame by the frame length to calculate the continuation time of the non-stationary signal (step S208). The continuation time measurement unit 13 then resets the count number C to 0.

On the other hand, when the vibration signal of the current frame is the non-stationary signal in step S207 (step S207-Yes), it is assumed that the current frame also corresponds to mastication or swallowing. Therefore, the continuation time measurement unit 13 increments the count number C of the frame representing the continuation time of the non-stationary signal by 1 (step S209). After step S208 or S209, the continuation time measurement unit 13 terminates the continuation time measurement processing of the non-stationary signal.

In step S205, the continuation time measurement unit 13 may store the time corresponding to the current frame in the memory unit 5 as a start time of the non-stationary signal. In step S208, the continuation time measurement unit 13 may calculate the continuation time of the non-stationary signal by subtracting the start time of the non-stationary signal from the time corresponding to the current frame. In this case, the processing of step S209 may be omitted.

Each time the period during which the non-stationary signal having a power above a certain level is continued ends, the continuation time measurement unit 13 outputs the continuation time calculated for the period to the determination unit 14.

The determination unit 14 determines whether the period corresponds to swallowing or mastication on the basis of the continuation time of the period during which the non-stationary signal having a power above a certain level is continued. As described above, the continuation time for swallowing is longer than that for mastication. Therefore, the determination unit 14 compares the continuation time of the period during which the non-stationary signal having a power above a certain level is continued with a predetermined time threshold, and determines that the period corresponds to swallowing when the continuation time is longer than the time threshold. On the other hand, the determination unit 14 determines that the period corresponds to mastication when the continuation time of the period, during which the non-stationary signal having a power above a certain level is continued, is shorter than the time threshold. The time threshold is set, for example, to 400 milliseconds, which is an intermediate length of the typical continuation time for swallowing and mastication.

Each time mastication or swallowing of the subject is detected, the determination unit 14 acquires the time of detection with reference to clock information included in the eating and drinking action detection apparatus. The determination unit 14 then stores the time when mastication or swallowing is detected in the memory unit 5 together with the determination result.

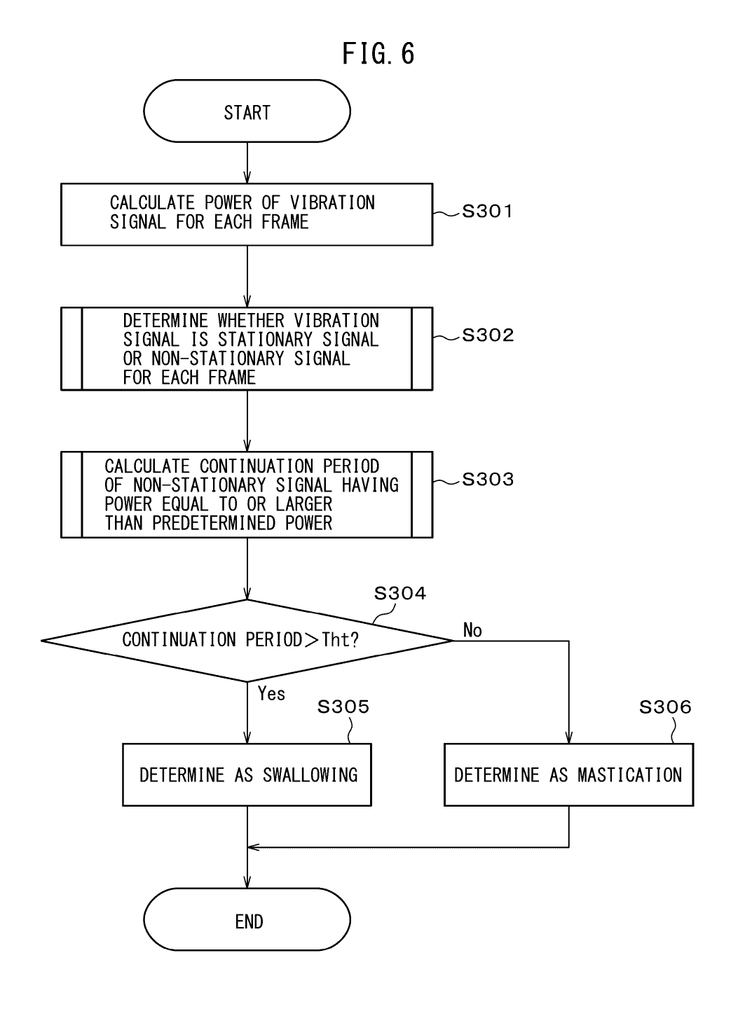

FIG. 6 is an operational flowchart of eating and drinking action detection processing. The power calculation unit 11 divides the vibration signal into each frame, and calculates power of the vibration signal for each frame (step S301). The stationarity determination unit 12 determines whether the vibration signal is the stationary signal or the non-stationary signal for each frame (step S302).

The continuation time measurement unit 13 obtains the length of the period during which the non-stationary signal having a power above a certain level is continued, on the basis of the determination results on the power of the vibration signal and on whether the vibration signal is the stationary signal or the non-stationary signal for each frame (step S303). The determination unit 14 determines whether the length of the period is longer than a time threshold Tht (step S304).

When the length of the period during which the non-stationary signal is continued is longer than the time threshold Tht (step S304-Yes), the determination unit 14 determines that the period corresponds to swallowing (step S305). On the other hand, when the length of the period during which the non-stationary signal is continued is equal to or shorter than the time threshold Tht (step S304-No), the determination unit 14 determines that the period corresponds to mastication (step S306). The processing unit 7 repeats the execution of the above-described eating and drinking action detection processing, until the vibration signal to be processed in the eating and drinking action detection processing is completed.

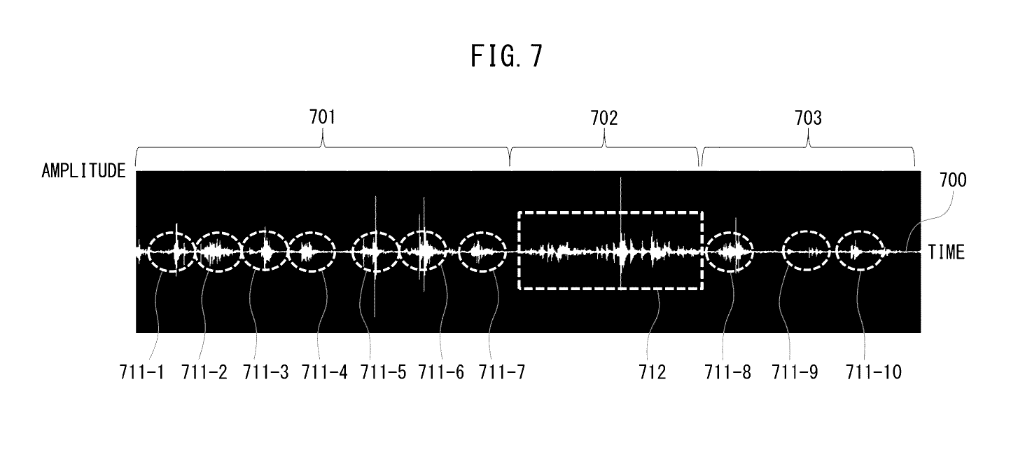

FIG. 7 is a drawing illustrating an example of a detection result when the eating and drinking action detection processing according to the present embodiment is applied to an audio signal acquired as a vibration signal. In FIG. 7, a horizontal axis represents time and a vertical axis represents amplitude of an audio signal 700. A section 701 and a section 703 are sections in which the subject is masticating, and a section 702 is a section in which the subject is swallowing. Sections 711-1 to 711-10 indicated by circles represent sections detected as mastication by applying the eating and drinking action detection processing according to the present embodiment. On the other hand, a section 712 indicated by a quadrangle represents a section detected as to be swallowing by applying the eating and drinking action detection processing according to the present embodiment.

As illustrated in FIG. 7, it is understood that swallowing and mastication are accurately identified and detected by the eating and drinking action detection processing according to the present embodiment, even when swallowing and mastication coexist.

As described above, the eating and drinking action detection apparatus analyzes a vibration signal corresponding to vibration produced from inside of the body of a subject to measure a period during which the non-stationary signal having a power above a certain level is continued, and determines whether the period corresponds to mastication or swallowing according to the length of the period. Therefore, even when a subject performs mastication and swallowing alternately, the eating and drinking action detection apparatus can detect mastication and swallowing and accurately identify mastication with swallowing. Further, the eating and drinking action detection apparatus makes it a condition that a vibration signal has a power above a certain level when detecting mastication or swallowing, which makes it possible to suppress a noise included in the vibration signal to be erroneously detected as mastication or swallowing.

Next, an eating and drinking action detection apparatus according to a second embodiment is described. The eating and drinking action detection apparatus according to the second embodiment adjusts a threshold to be used in stationarity determination, a power threshold to be used in continuation time determination, or a time threshold to be used for identifying swallowing with mastication, on the basis of the determination result of swallowing and mastication during a certain period.

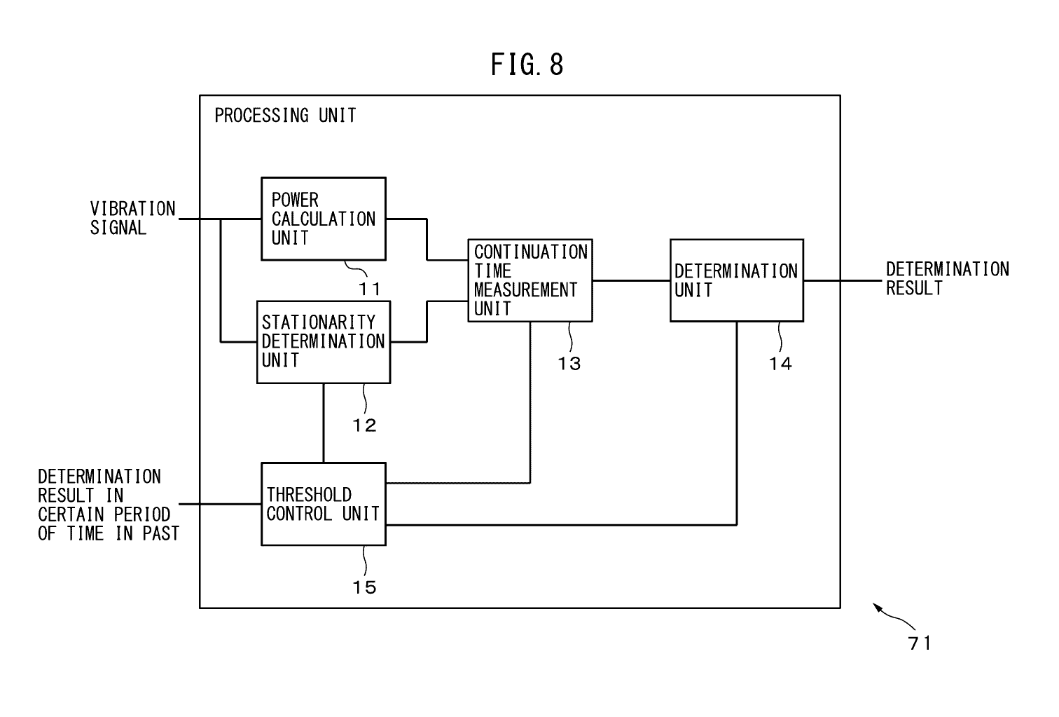

FIG. 8 is a functional block diagram of a processing unit of the eating and drinking action detection apparatus according to the second embodiment. A processing unit 71 of the eating and drinking action detection apparatus according to the second embodiment includes a power calculation unit 11, a stationarity determination unit 12, a continuation time measurement unit 13, a determination unit 14, and a threshold control unit 15. The eating and drinking action detection apparatus according to the second embodiment is different in comparison with the eating and drinking action detection apparatus according to the first embodiment in the point that the processing unit 71 includes a threshold control unit 15. Therefore, the threshold control unit 15 and the relevant parts are described below. With respect to other components of the eating and drinking action detection apparatus according to the second embodiment, refer to the explanation of the corresponding components of the eating and drinking action detection apparatus according to the first embodiment.

The threshold control unit 15 adjusts the threshold to be used in the stationarity determination, the power threshold to be used in the continuation time determination, or the time threshold to be used for identifying swallowing with mastication, when it is assumed that the determination result of swallowing and mastication during a certain period in the past is abnormal.

For example, when no swallowing is detected during a certain period of about several hours or one day, it is assumed that the eating and drinking action detection apparatus failed in detecting of swallowing of a subject, or erroneously detected swallowing of a subject as mastication. In such a case, it is preferable that at least any of the above-described thresholds is adjusted by the threshold control unit 15, so that swallowing may become easy to be detected or mastication may become difficult to be detected. For example, the threshold control unit 15 decreases the power threshold to be used in the continuation time determination, increases the threshold to be used in the stationarity determination, or decreases the time threshold to be used for identifying swallowing with mastication.

On the other hand, the number of times of swallowing detected during a certain period of about several hours or one day is too many, it is assumed that a noise is erroneously detected as a signal representing eating and drinking of a subject, or mastication of a subject is erroneously detected as swallowing. Therefore, in such a case, it is preferable that at least any of the above-described thresholds is adjusted by the threshold control unit 15, so that mastication may become easy to be detected or a noise may become difficult to be detected as an eating and drinking action. For example, the threshold control unit 15 increases the power threshold used in the continuation time determination, decreases the threshold used in the stationarity determination, or increases the time threshold used for identifying swallowing with mastication.

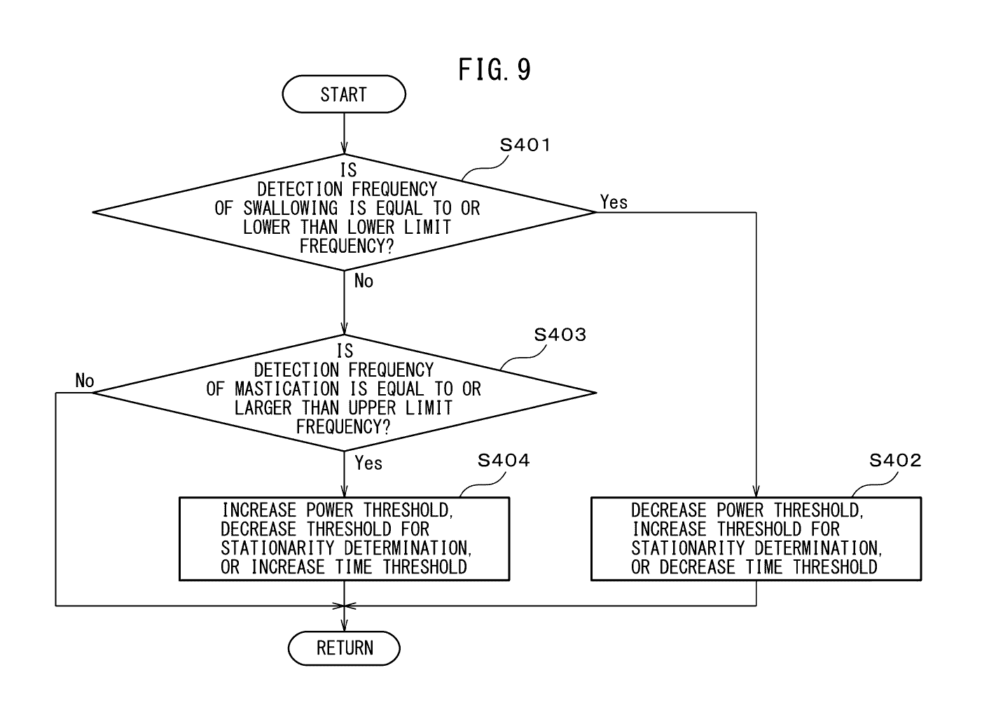

FIG. 9 is an operational flowchart of threshold control processing by the threshold control unit 15. The threshold control unit 15 executes threshold control processing in accordance with the following operational flowchart, each time eating and drinking action detection processing is executed for a certain period (for example, several hours to one day).

The threshold control unit 15 determines whether the detection frequency of swallowing is equal to or lower than a predetermined lower limit frequency, among the frequency of swallowing and mastication detected during a certain period (step S401). Note that the lower limit frequency is set to 0.01, for example.

When the detection frequency of swallowing is equal to or lower than the lower limit frequency (step S401-Yes), it is assumed that swallowing of a subject is not appropriately detected. Therefore, the threshold control unit 15 decreases the power threshold used in the continuation time determination, increases the threshold used in the stationarity determination, or decreases the time threshold used for identifying swallowing with mastication (step S402). Note that in one adjustment of a threshold, the threshold control unit 15 may decrease the power threshold, for example, by 1 dB. Alternatively, the threshold control unit 15 may increase the threshold against the maximum value of autocorrelation to be used in the stationarity determination, for example, by 0.01. Alternatively, the threshold control unit 15 may decrease the time threshold used for identifying swallowing with mastication, for example, by 25 milliseconds. Note that the threshold control unit 15 may change any one or any two of the thresholds by turns, or all of the thresholds, each time the processing of step S402 is performed.

On the other hand, when the detection frequency of swallowing is higher than the lower limit frequency in step S401 (step S401-No), the threshold control unit 15 determines whether the detection frequency of swallowing is equal to or higher than a predetermined upper limit frequency, among the frequency of swallowing and mastication detected during a certain period (step S403). Note that the upper limit frequency is set to 0.1, for example.

When the detection frequency of swallowing is equal to or higher than the upper limit frequency (step S403-Yes), it is assumed that the vibration produced by a factor other than swallowing of a subject is erroneously detected as swallowing. Therefore, the threshold control unit 15 may increase the power threshold used in the continuation time determination, decreases the threshold used in the stationarity determination, or increases the time threshold used for identifying swallowing with mastication (step S404). Also in this case, in one adjustment of a threshold, the threshold control unit 15 may change each of the thresholds by the amount of the same degree as the amount of adjustments in step S402. The threshold control unit 15 may change any one of these thresholds, or any two of the thresholds by turns, or all of the thresholds, each time processing of step S404 is performed. In step 403, when the detection frequency of swallowing is lower than the upper limit frequency (step S403-No), the threshold control unit 15 does not correct the above-described thresholds.

The threshold control unit 15 may decrease an amount of adjustment in one adjustment of a threshold, as the number of executed processing of step S402 or S404 increases. Hereby, the threshold control unit 15 can prevent threshold control processing from being repeated infinitely due to the amount of adjustment of a threshold is in excess.

According to the second embodiment, the eating and drinking action detection apparatus automatically adjusts each threshold used by the eating and drinking action detection processing corresponding to the detection frequency of swallowing during a certain period in the past, and therefore it is possible to appropriately detect swallowing and mastication of a subject corresponding to the environment where the eating and drinking action detection apparatus is used.

Next, an eating and drinking action detection apparatus according to a third embodiment is described. The eating and drinking action detection apparatus according to the third embodiment determines, each time swallowing of a subject is detected, whether the subject is masticating for more than an appropriate number of times corresponding to the number of mastication performed just before swallowing.

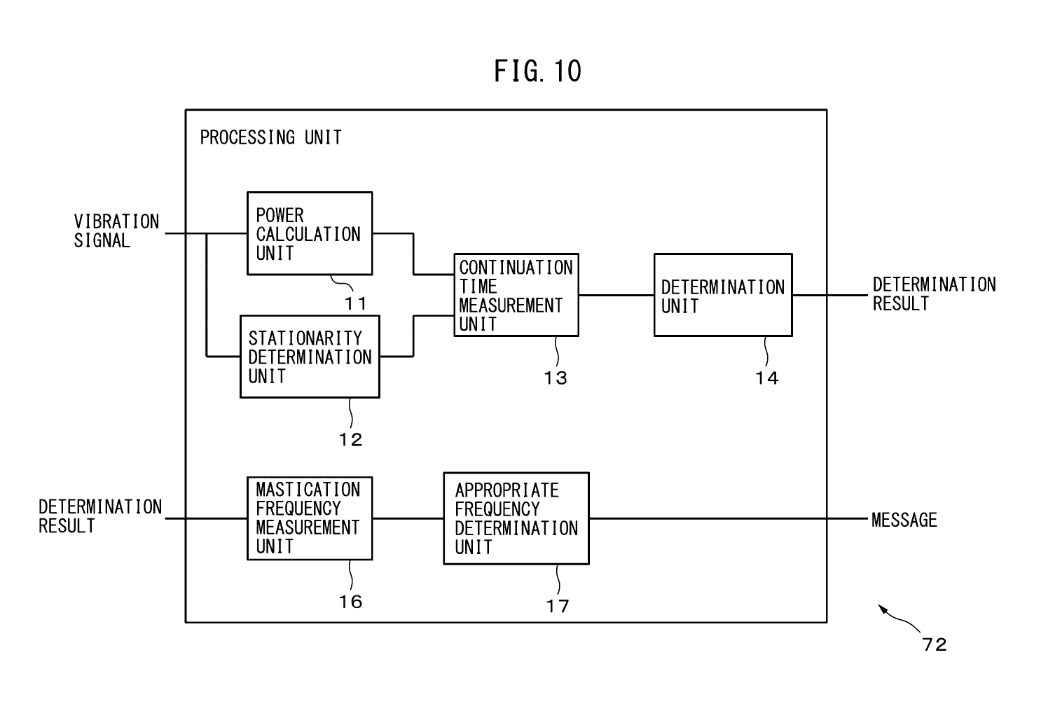

FIG. 10 is a functional block diagram of a processing unit of the eating and drinking action detection apparatus according to the third embodiment. The processing unit 72 of the eating and drinking action detection apparatus according to the third embodiment includes a power calculation unit 11, a stationarity determination unit 12, a continuation time measurement unit 13, a determination unit 14, a mastication frequency measurement unit 16, and an appropriate frequency determination unit 17. The eating and drinking action detection apparatus according to the third embodiment is different in comparison with the eating and drinking action detection apparatus according to the first embodiment in that the processing unit 72 includes the mastication frequency measurement unit 16 and the appropriate frequency determination unit 17. Therefore, the mastication frequency measurement unit 16, the appropriate frequency determination unit 17 and the relevant parts are described below. With respect to other components of the eating and drinking action detection apparatus according to the third embodiment, refer to the explanation of the corresponding components of the eating and drinking action detection apparatus according to the first embodiment.

After the previous swallowing of a subject is detected, the mastication frequency measurement unit 16 increments, each time mastication of a subject is detected by the determination unit 14, a count value representing the number of the detections of mastication. When swallowing of the subject is detected by the determination unit 14, the mastication frequency measurement unit 16 passes the count value representing the number of the detections of mastication to the appropriate frequency determination unit 17 as the number of mastication performed by the subject between the successive swallowing. Thereafter, the mastication frequency measurement unit 16 resets the count value to 0.

The appropriate frequency determination unit 17 compares the number of mastication performed by the subject between the successive swallowing with a predetermined mastication number threshold (for example, 20 times). The appropriate frequency determination unit 17 causes a message to be displayed on the user interface unit 4 to inform the subject of a small number of mastication, when the number of mastication is lower than the mastication number threshold. Alternatively, when the eating and drinking action detection apparatus includes a loudspeaker (not shown), the appropriate frequency determination unit 17 may cause an audio signal to be outputted to inform the subject of the small number of mastication from the loudspeaker.

The eating and drinking action detection apparatus according to the third embodiment obtains the number of mastication per swallowing of the subject to give advice to the subject regarding mastication depending on the result.

Next, an eating and drinking action detection apparatus according to a fourth embodiment is described. The eating and drinking action detection apparatus according to the fourth embodiment determines whether a subject eats orderly corresponding to whether the time when mastication and swallowing of the subject are detected satisfies a predetermined regularity condition.

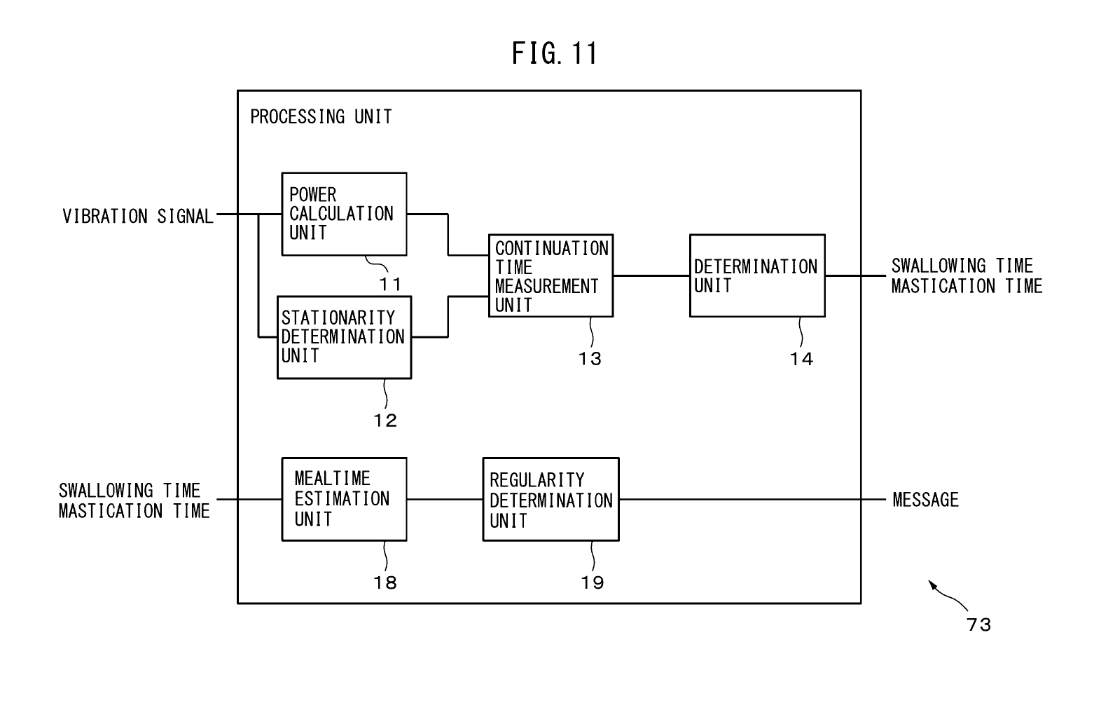

FIG. 11 is a functional block diagram of a processing unit of the eating and drinking action detection apparatus according to the fourth embodiment. The processing unit 73 of the eating and drinking action detection apparatus according to the fourth embodiment includes a power calculation unit 11, a stationarity determination unit 12, a continuation time measurement unit 13, a determination unit 14, a mealtime estimation unit 18, and a regularity determination unit 19. The eating and drinking action detection apparatus according to the fourth embodiment is different in comparison with the eating and drinking action detection apparatus according to the first embodiment in the point that the processing unit 73 includes the mealtime estimation unit 18 and the regularity determination unit 19. Therefore, the mealtime estimation unit 18, the regularity determination unit 19 and relevant parts are described below. With respect to other components of the eating and drinking action detection apparatus according to the fourth embodiment, refer to the explanation of the corresponding components of the eating and drinking action detection apparatus according to the first embodiment.

Each time mastication or swallowing of the subject is detected, the determination unit 14 acquires the time of detection with reference to clock information included in the eating and drinking action detection apparatus. The determination unit 14 stores the time when mastication or swallowing is detected, and continuation time thereof to the memory unit 5.

The mealtime estimation unit 18 estimates the time when the subject has a meal on the basis of the time when mastication is detected and the time when swallowing is detected for every day, every several days, or every specific day of the week.

For example, the mealtime estimation unit 18 calculates, for each of a certain period (for example, 30 minutes), the total of the continuation time of mastication detected during the period and the total of the continuation time of swallowing detected. The mealtime estimation unit 18 determines that the subject had a meal during the certain period, when a ratio of the total of the continuation time of mastication in a certain period is 50% or more and a ratio of the total of the continuation time of swallowing in the certain period is 1% or more.

The mealtime estimation unit 18 determines the period in which the sum of the total of the continuation time of mastication and the total of the continuation time of swallowing is the maximum among periods determined that the subject had a meal in the periods before predetermined time in the morning (for example, 11:00 a.m.), as estimated time when the subject had a breakfast. Similarly, the mealtime estimation unit 18 determines the period in which the sum of the total of the continuation time of mastication and the total of the continuation time of swallowing is the maximum among periods determined that the subject had a meal in the periods around noon (for example, 11:00 a.m. to 3:00 p.m.), as estimated time when the subject had a lunch. Further, the mealtime estimation unit 18 determines the period in which the sum of the total of the continuation time of mastication and the total of the continuation time of swallowing is the maximum among periods determined that the subject had a meal in the periods after predetermined time in the afternoon (for example, after 3:00 p.m.), as estimated time when the subject had a dinner.

The mealtime estimation unit 18 stores the estimated time of the breakfast, the lunch, and the dinner of the subject in the memory unit 5.

The regularity determination unit 19 determines whether the subject has regular eating habit on the basis of whether a statistic of distribution of the estimated daily mealtime for the subject in a certain period (for example, for one week or one month) satisfies the predetermined regularity condition. For example, the regularity determination unit 19 calculates a standard deviation of the dinner time of the subject during the certain period as the statistic of distribution of the estimated daily mealtime for the subject. When the standard deviation is larger than a certain value (for example, 1 hour), the regularity determination unit 19 determines that the regularity condition is not satisfied and the subject does not have regular eating habit. Alternatively, the regularity determination unit 19 calculates a ratio of the dinner time of the subject after predetermined time (for example, 10:00 p.m.) during the certain period as the statistic of distribution of the estimated daily mealtime for the subject. When the ratio is equal to or larger than a certain value (for example, 80%), the regularity determination unit 19 may determine that the regularity condition is not satisfied and the subject does not have regular eating habit.

When the regularity determination unit 19 determines that the subject does not have regular eating habit, the regularity determination unit 19 causes a message to be displayed on the user interface unit 4 to inform the subject that the subject irregularly has a meal.

The eating and drinking action detection apparatus according to the fourth exemplary embodiment can determine the regularity of the meal of the subject to give advice to the subject regarding the regularity of the meal depending on the result.

The second to fourth embodiments may be combined.

In above-described embodiments or modified example, the subject may not be limited to a human but may be an animal such as livestock.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present inventions have been described in detail, it should be understood that the various changes, substitutions, and alterations could be made hereto without departing from the spirit and scope of the invention.

What is claimed is:

1. An eating and drinking action detection apparatus comprising:
a vibration sensor configured to acquire vibration produced from inside of a body of a subject and generate a vibration signal corresponding to the vibration; and
a processor configured to:
divide the vibration signal into each frame with a predetermined time length to calculate power of the vibration signal for each frame;
determine, for each frame, whether the frame is a stationary signal having a periodicity or a non-stationary signal having no periodicity;
detect, based on the power of each frame and a determination result for each frame whether the frame is the stationary signal or the non-stationary signal, a period of the non-stationary signal being continued while the power of the vibration signal is equal to or larger than a power threshold;
acquire a continuation time of the period; and
determine, based on the continuation time, whether the subject performed swallowing or mastication in the period of the non-stationary signal being continued.

2. The eating and drinking action detection apparatus according to claim 1, wherein determination of whether the subject performed swallowing or mastication determines that the subject performed swallowing in the period of the non-stationary signal being continued, when the continuation time is longer than a time threshold, while determining that the subject performed mastication in the period of the non-stationary signal being continued, when the continuation time is equal to or shorter than the time threshold.

3. The eating and drinking action detection apparatus according to claim 2, wherein the processor is further configured to obtain a feature quantity representing a periodicity for each frame of the vibration signal and wherein determination of whether the frame is the stationary signal or the non-stationary signal determines that the frame is the stationary signal, when the feature quantity is equal to or larger than a stationarity determination threshold, while determining that the frame is the non-stationary signal, when the feature quantity is smaller than the stationarity determination threshold.

4. The eating and drinking action detection apparatus according to claim 3, further comprising:
a memory configured to store detection time of swallowing; and wherein
the processor is further configured to calculate detection frequency of swallowing in a predetermined period, with reference to the detection time of swallowing, and adjust at least one of the power threshold, the time threshold, and the stationary determination threshold in response to the frequency.

5. The eating and drinking action detection apparatus according to claim 4, wherein adjusting at least one of the power threshold, the time threshold, and the stationary determination threshold executes at least one of reduction of the power threshold, increase of the stationary determination threshold, and reduction of the time threshold, when the frequency is equal to or lower than a predetermined lower limit.

6. The eating and drinking action detection apparatus according to claim 4, wherein adjusting at least one of the power threshold, the time threshold, and the stationary determination threshold executes at least one of increase of the power threshold, reduction of the stationary determination threshold, and increase of the time threshold, when the frequency is equal to or larger than a predetermined upper limit.

7. The eating and drinking action detection apparatus according to claim 1, wherein the processor is further configured to:
count number of detections of mastication until swallowing is detected; and
make notification when the number of detections of mastication at the time when swallowing is detected is smaller than a predetermined number.

8. The eating and drinking action detection apparatus according to claim 1, further comprising:
a memory configured to store time of detection of swallowing and a continuation time of a period of the non-stationary signal corresponding to swallowing being continued, as well as time of detection of mastication and a continuation time of a period of the non-stationary signal corresponding to mastication being continued; and wherein the processor is further configured to:
estimate time when the subject had a meal, based on the time of the detection of swallowing and the continuation time of the period of the non-stationary signal corresponding to swallowing being continued, as well as the time of the detection of mastication and the continuation time of the period of the non-stationary signal corresponding to mastication being continued; and
make notification when a statistic value of distribution of the estimated time when the subject had a meal does not satisfy a predetermined regularity condition during a certain period.

9. An eating and drinking action detection method comprising:
acquiring vibration produced from inside of a body of a subject and generating a vibration signal corresponding to the vibration;
dividing the vibration signal into each frame with a predetermined time length to calculate power of the vibration signal for each frame;
determining, for each frame, whether the frame is a stationary signal having a periodicity or a non-stationary signal having no periodicity;
detecting, based on the power of each frame and a determination result for each frame whether the frame is the stationary signal or the non-stationary signal, a period of the non-stationary signal being continued while the power of the vibration signal is equal to or larger than a power threshold;
acquiring a continuation time of the period; and
determining, based on the continuation time, whether the subject performed swallowing or mastication in the period of the non-stationary signal being continued.

10. The eating and drinking action detection method according to claim 9, wherein determination of whether the subject performed swallowing or mastication determines that the subject performed swallowing in the period of the non-stationary signal being continued, when the continuation time is longer than a time threshold, while determining that the subject performed mastication in the period of the non-stationary signal being continued, when the continuation time is equal to or shorter than the time threshold.

11. The eating and drinking action detection method according to claim 10, further comprising:
obtaining a feature quantity representing a periodicity for each frame of the vibration signal, and wherein determination of whether the frame is the stationary signal or the non-stationary signal determines that the frame is the stationary signal, when the feature quantity is equal to or larger than a stationarity determination threshold, while determining that the frame is the non-stationary signal, when the feature quantity is smaller than the stationarity determination threshold.

12. The eating and drinking action detection method according to claim 11, further comprising:
storing detection time of swallowing in a memory;
calculating detection frequency of swallowing in a predetermined period, with reference to the detection time of swallowing; and
adjusting at least one of the power threshold, the time threshold, and the stationary determination threshold in response to the frequency.

13. The eating and drinking action detection method according to claim 12, wherein adjusting at least one of the power threshold, the time threshold, and the stationary determination threshold executes at least one of reduction of the power threshold, increase of the stationary determination threshold, and reduction of the time threshold, when the frequency is equal to or lower than a predetermined lower limit.

14. The eating and drinking action detection method according to claim 12, wherein adjusting at least one of the power threshold, the time threshold, and the stationary determination threshold executes at least one of increase of the power threshold, reduction of the stationary determination threshold, and increase of the time threshold, when the frequency is equal to or larger than a predetermined upper limit.

15. The eating and drinking action detection method according to claim 9, further comprising:
counting number of detections of mastication until swallowing is detected; and
making notification when the number of detections of mastication at the time when swallowing is detected is smaller than a predetermined number.

16. The eating and drinking action detection method according to claim 9, further comprising:
storing time of detection of swallowing and a continuation time of a period of the non-stationary signal corresponding to swallowing being continued, as well as time of detection of mastication and a continuation time of a period of the non-stationary signal corresponding to mastication being continued in a memory;
estimating time when the subject had a meal, based on the time of the detection of swallowing and the continuation time of the period of the non-stationary signal corresponding to swallowing being continued, as well as the time of the detection of mastication and the continuation time of the period of the non-stationary signal corresponding to mastication being continued; and
making notification when a statistic value of distribution of the estimated time when the subject had a meal does not satisfy a predetermined regularity condition during a certain period.

17. A non-transitory computer-readable recording medium having recorded thereon a computer program for detecting eating and drinking action that causes a computer to execute a process comprising:
dividing a vibration signal corresponding to vibration produced from inside of a body of a subject into each frame with a predetermined time length to calculate power of the vibration signal for each frame;
determining, for each frame, whether the frame is a stationary signal having a periodicity or a non-stationary signal having no periodicity;
detecting, based on the power of each frame and a determination result for each frame whether the frame is the stationary signal or the non-stationary signal, a period of the non-stationary signal being continued while the power of the vibration signal is equal to or larger than a power threshold;
acquiring a continuation time of the period; and
determining, based on the continuation time, whether the subject performed swallowing or mastication in the period of the non-stationary signal being continued.
